(19) United States
(12) Patent Application Publication
(10) Pub. No.: US 2026/0294500 A1
(43) Pub. Date: Oct. 1, 2026

(54) Catheter with multiple cryotherapy delivery sites

(71) Applicant: Mina Padula, London (GB)

(72) Inventor: Mina Padula, London (GB)

(21) Appl. No.: 19/067,859

(22) Filed: Mar. 1, 2025

Related U.S. Application Data
(60) Provisional application No. 63/560,750, filed on Mar. 3, 2024.

(51) Int. Cl. A61B 18/02; A61B 18/00; A61B 18/14

(52) U.S. Cl. CPC A61B 18/02; A61B 18/1492; A61B 2018/00178; A61B 2018/00184; A61B 2018/00351; A61B 2018/00577; A61B 2018/00714; A61B 2018/0212; A61B 2018/0262; A61B 2018/1407; A61B 2018/1467

(57) ABSTRACT

A catheter includes a tip portion and a therapy portion. The tip portion is configured to deliver cryotherapy. The therapy portion extends from the tip portion. The therapy portion includes one or more segments. Each segment is configured to deliver cryotherapy. Electrodes are disposed on an outer surface of the therapy portion.

### CROSS-REFERENCE TO RELATED APPLICATIONS

The present application claims the benefit of U.S. Provisional Patent Application No. 65/560,750, filed on Mar. 3, 2024, which is hereby incorporated by reference in its entirety.

### FIELD OF THE INVENTION

The present system pertains to the ablation of tissue by exposing it to cold.

### BACKGROUND OF THE INVENTION

Disturbances of heart rhythm may result from the emergence of abnormal depolarization from areas of diseased heart muscle or from the propagation of waves of depolarization in a recirculating manner around the heart muscle. Ablation of heart muscle is used to create areas of scarring to cure heart rhythm disturbances, either by encircling an abnormal area or by blocking the route through which depolarization can recirculate. This can be accomplished by cryotherapy in which the tissue is destroyed by freezing or by electrical injury in the form of radiofrequency or pulsed field energy. In many cases, cure of an arrhythmia requires one or more continuous line of blocked conduction. A line of block constructed as a series of points is time-consuming and technically difficult. A single lesion that forms a continuous line is difficult to achieve reliably with current equipment; the movement of the heart and the uneven contour of its surface make it difficult to keep a catheter in continuous contact over distance.

Cryotherapy may be achieved by the flow of a coolant through the catheter, including nitrous oxide which enters the catheter as a liquid and cools by transitioning to a gas within the catheter tip, or nitrogen introduced as a liquid or as a near critical fluid and exiting as a gas.

Cryotherapy catheters act by freezing tissue in contact with the catheter to a temperature below the freezing point of blood and tissue. This produces a zone around the catheter in which the blood and the tissue are frozen solid as the water in the blood and tissue are turned to ice. This is informally referred to as an “ice ball” and can extend for several millimeters into the tissue. The catheter therefore becomes adherent to the tissue, but this attachment is weak as most catheters have a smooth surface.

### SUMMARY OF THE INVENTION

Examples of the present technology include a catheter long enough to reach from the point of puncture of the vein to any point in the heart and narrow enough to fit through the veins and through a puncture in the interatrial septum. The end of the catheter that is placed in the heart delivers cryotherapy at its tip and independently in one or more segments adjacent to the tip or to each other. The other end has a handle to be grasped by the operator to advance, withdraw or apply torque force to the catheter. The parts of the catheter located farther from the handle are referred to as “distal”; areas closer to the handle are termed “proximal”. The catheter may have a deflection wire to allow adjustment of the curve close to the tip of the catheter and may have a central channel through which a stylet with a pre-formed curve and a chosen level of stiffness can be advanced to control the curvature and stiffness of the catheter. The catheter may be advanced through a deflectable sheath as an alternative method of directing it to a desired location. The tip of the catheter may include an electrode or array of electrodes with a surface that may be textured or ridged to enhance adherence to the ice-ball that forms around it when cryotherapy is administered through it. Using this adherence as an anchor, the other cryotherapy segment or segments may be forced into contact with the tissue to deliver cryotherapy in a continuous line.

### BRIEF DESCRIPTION OF THE DRAWINGS

FIG. 1 illustrates an example of a catheter.

FIG. 2 illustrates an example of a catheter tip.

FIG. 3 illustrates an example structure of a catheter tip.

FIG. 4 illustrates electrical elements of a catheter tip.

FIG. 5 illustrates internal elements of a catheter.

FIGS. 6a-b illustrate an example of using a catheter.

FIGS. 7a-b illustrate another example of using a catheter.

### DETAILED DESCRIPTION

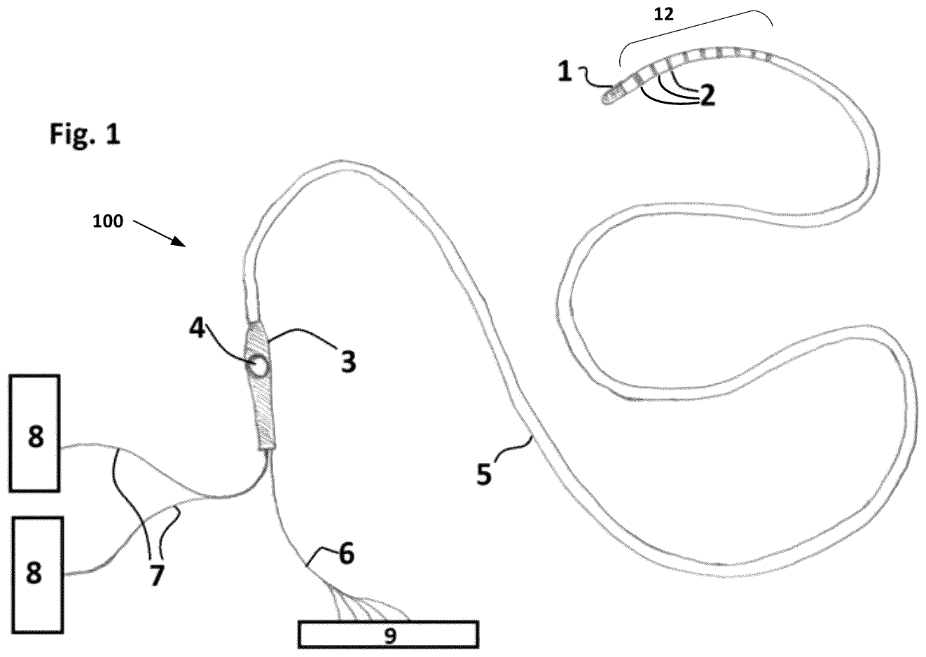

FIG. 1 shows a catheter 100 with its connection to corresponding consoles to deliver coolant to create extremely low temperatures at and near the tip of the catheter through it and display electrical data from it. The catheter tip portion (1) is intended to lie in the patient's body in contact with the tissue to be ablated. The section of the catheter adjacent to the tip portion 1, therapy portion 12, has a number of electrodes (2) each consisting of a metal ring extending circumferentially around therapy portion 12 with the metal surface exposed to record electrical activity from the tissue and deliver electricity in the form of pulsed field and/or radiofrequency energy or otherwise provide therapy to contacted tissue and/or tissue near the area of contact. The body 5 of the catheter 100 may have the same consistency as the tip portion 1 and therapy portion 200 (e.g., a soft consistency) to facilitate advancement through the blood vessels. The handle (3) remains external to the patient's body and is designed to allow the operator to grip the catheter 100 to apply force to advance, withdraw or rotate it. The handle 3 may contain a control (4) attached to a deflection-wire within the catheter 100 to allow the operator to adjust the curve of the catheter 100 and thereby adjust the location of tip 1, and/or the operator may advance a stylet through a central lumen of the catheter to achieve the same effect. Emerging from the handle there are attachments (7) to consoles (8) for the delivery of coolant to the cryotherapy elements of the catheter, and an electrical connector 9 (or connectors) to connect to a system for processing and displaying electrical signals recorded from an electrode in tip portion (1) and electrodes in therapy portion 12 (e.g., electrodes 2). The cryotherapy delivery systems for the multiple segments (e.g., segments in therapy portion 12) are capable of independent activation but may be aggregated into a single machine. The electrical cable 6 may also be used to deliver pulsed field or radiofrequency energy through one or more electrode in tip portion (1) and/or one or more electrodes in therapy portion 12 (e.g., electrodes 2.

A cryotherapy element located in the tip portion 1 may be referred to as a first cryotherapy element, or distal cryotherapy element. A cryotherapy element adjacent to tip portion 1 in therapy portion 12 is referred to as “a second cryotherapy element”. Any additional cryotherapy element may be similarly numbered in sequence from distal to proximal.

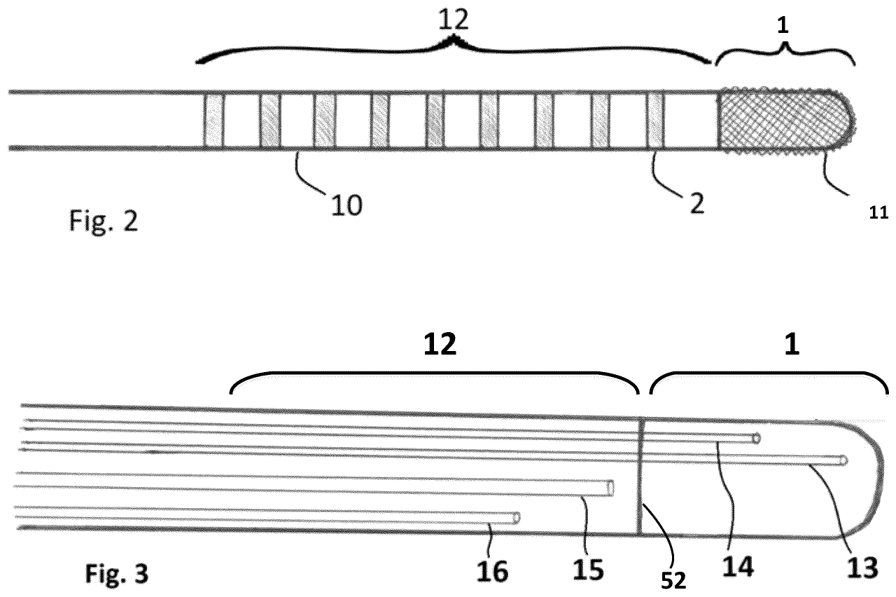

FIG. 2 is a more detailed view of the tip portion 1 and therapy portion 12 of the catheter 100 including a large distal metallic electrode or array of electrodes (11) which has a textured, corrugated or ridged surface to enhance adherence to surrounding ice or frozen tissue when frozen to colder than the freezing point of blood and tissue. This distal electrode or array of electrodes 11 contains the distal cryotherapy element (cooling element), providing the ability to cool tip portion 1 of the catheter 100 to very low temperatures by delivering a coolant consisting of a liquid or a near critical fluid. This provides cooling that is distributed all over the distal electrode or array (11). The adjacent segment of the catheter, therapy portion 12 (10) is of a soft material giving flexibility. Metal electrodes 2 are arranged as rings that extend circumferentially around the soft material of the catheter. The therapy portion 12 may include one or more additional cryotherapy elements (cooling elements) capable of freezing all along therapy portion 12 to very low temperatures independent of the operation of any cryotherapy element in tip portion 1. Additionally, where therapy portion 12 includes two or more cryotherapy elements, each such cryotherapy element may be independently controllable (e.g., to enable freezing of selected segments of therapy portion 12, without freezing other segments).

FIG. 3 is a longitudinal section of the tip portion 1 and therapy portion 12 showing certain components involved in delivering cryotherapy. The tip portion (1) is sealed from therapy portion 12 by a barrier 52 to form a sealed chamber within tip portion 1. A tube (13) delivers coolant as a liquid or near critical fluid to tip portion 1 and a second tube (14) extends into tip portion 1 to remove the coolant (e.g., in gaseous form). The section of the therapy portion 12 that is adjacent to the tip portion 1 also has a tube (15) to deliver coolant and a tube to remove it (16). If there is a third, fourth or subsequent cooling segment in therapy portion 12, these will be separated from the cooling segment adjacent to the tip electrode and each will have its own pair of tubes for delivery and removal of coolant. For example, a wall or barrier (similar to barrier 52 may separate each segment from a neighboring segment to form separate chambers in which coolant may flow, with each such chamber having a dedicated supply tube and a dedicated drain tube to enable individual control of coolant on a segment-by-segment basis.

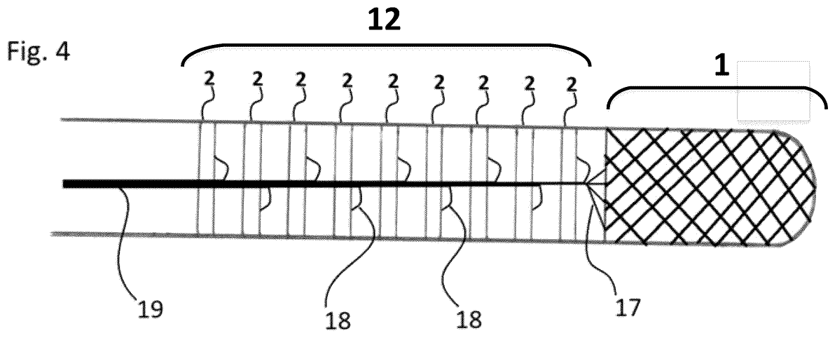

FIG. 4 is a longitudinal section of the end of the catheter 100 including tip portion 1 and therapy portion 12 showing the components that record electrical data and deliver electrical therapy. In cases where the tip portion (1) consists of a single electrode, a single insulated wire will extend from it to the handle of the catheter. If tip portion 1 includes a plurality (e.g., an array) of electrodes, there will be multiple insulated wires (17) linking each electrode (e.g., each component of the array) to the catheter handle 3. The ring electrodes (2) on the outer surface of therapy portion 12 each have an insulated wire 18 to connect to the catheter handle 3. The wires from the tip electrode(s) and ring electrodes 2 may be gathered into a single bundle 19 as show or may extend individually.

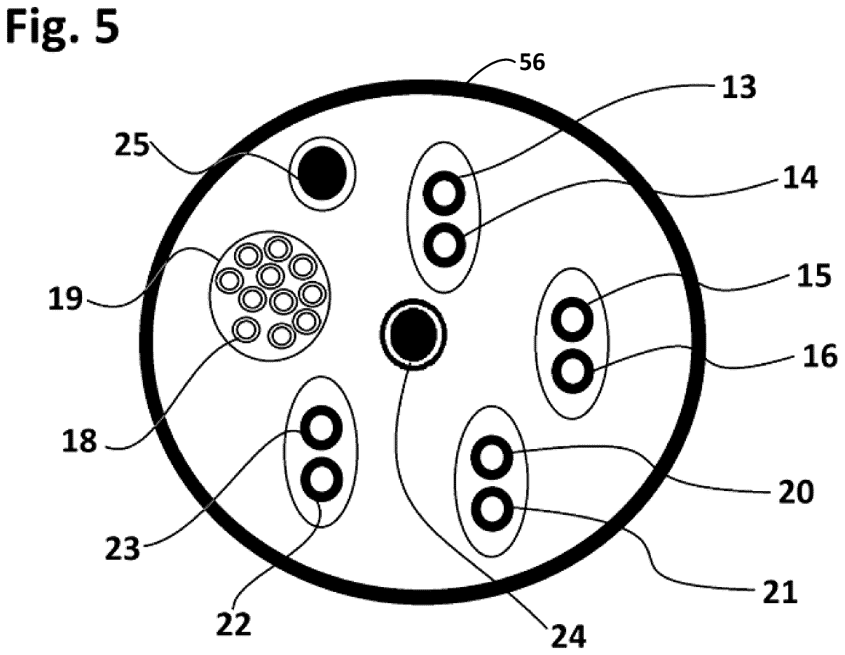

FIG. 5 is a cross section of the catheter 100 near the handle 3. The outer layer 56 of the catheter is a waterproof flexible material to protect the components from the bloodstream. The space inside this contains a bundle of cryotherapy elements for each freezing section of the catheter. The bundle for the tip electrode or array consists of a tube for coolant delivery (13) and a tube for coolant removal (14) which may be gathered together or lie independently within the insulation. The more proximal cooling segment or segments in therapy portion 12 each similarly has a tube for coolant delivery (15, 20, 22) and a tube for coolant removal (16, 21, 23). The electrical elements of the end of the catheter are each connected to the handle by an insulated wire (18), and these may be gathered into a bundle (19) or lie free in the catheter. There may be a central lumen in the catheter (24) through which a pre-formed stylet can be advanced to near the tip to confer a chosen shape. There may also be a deflection wire (25) running through the catheter and connected near the tip to provide adjustable deflection of the tip.

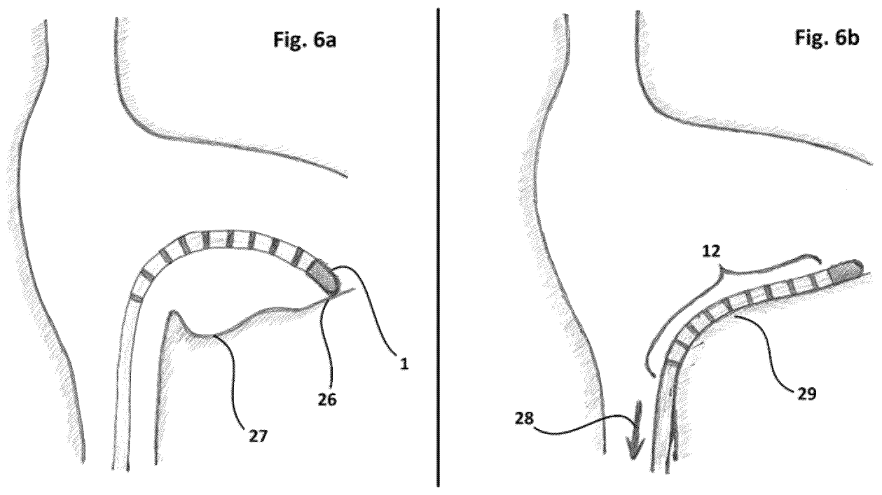

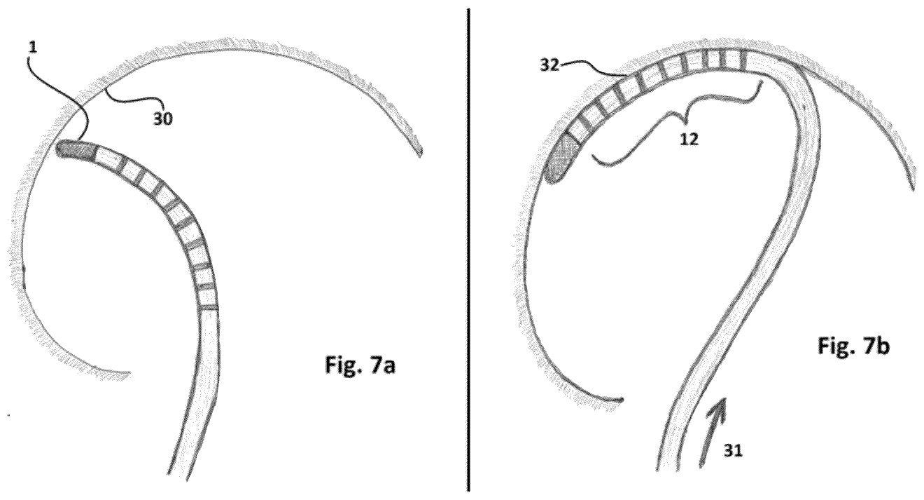

FIGS. 6a-b demonstrate an example mode of use of the catheter 100 in an area of the targeted tissue surface that is convex in shape (27). The initial placement is shown in FIG. 6a, where the catheter tip portion (1) is placed in firm contact with the tissue (26) at one end of the line along which the operator intends to achieve block of conduction. Cryotherapy is delivered at the tip portion 1 (e.g., by flowing coolant through tube 13 to a cavity in tip portion 1 and removing the coolant through tube 14), creating a ball of ice that extends into the tissue. Because of the textured, corrugated or ridged surface of tip portion 1, this ice-ball anchors tip portion 1 firmly to the tissue 26. The next step is shown in FIG. 6b. Traction (28) is applied from the handle of the catheter, transmitting the force to the tip. With the tip anchored in place, the adjacent part of the catheter, including therapy portion 12, is pulled into firm contact with the tissue all along the length of the intended line (29), the force evening out any irregularity in the contour. Cryotherapy may then be delivered throughout the length of therapy portion 12 or to one or more selected segment(s) of therapy portion 12. Once tip portion 1 is fully attached, tissue ablation may be enhanced by the delivery of pulsed field energy through all electrodes (e.g., electrodes 2 of therapy portion 12).

FIGS. 7a-b illustrate the use of the catheter in an area of the targeted tissue surface that is convex in shape (30). In the first step (FIG. 7a) the catheter is advanced to a position that places the tip portion (1) in firm contact with the tissue at one end of the intended line of block. Cryotherapy delivered through the tip portion 1 forms an ice-ball that ablates this tissue and causes firm adherence of the catheter to the tissue, enhanced by the rough or ridged surface of tip portion 1. The next step (FIG. 7b) is to apply forward force (31) to the catheter from the handle, causing the catheter, including therapy portion 12, to curve into firm contact with the tissue (32). Cryotherapy is then delivered throughout therapy portion 12 or selected segment(s) of therapy portion 12 producing a lesion that is continuous with that produced by the distal cryotherapy element in tip portion 1.

An example catheter includes 2 or more areas capable of delivering cryotherapy. The cryotherapy elements may include one in the tip of the catheter and one or more that lie proximally to this. The cryotherapy elements are continuous one with the next.

The example catheter may be of a uniform flexible consistency to facilitate its curvature against the heart wall with gentle force. The operator can control the position of the catheter by applying forward force or traction, by applying torque through rotation of the handle in a clockwise or anticlockwise manner, by inserting a pre-formed stylet, by applying force on a deflection wire controlled from the handle and/or by adjusting the curvature of a deflectable sheath through which the catheter may be delivered to the cardiac chamber.

An example catheter has a distal electrode or array of electrodes that constitute the surface of the distal cryotherapy element. There are electrodes on the more proximal cryotherapy element or elements. Each electrode transmits electrical activity through an insulated wire in the catheter to an electrical output cable attached to the handle of the catheter that transmits it to a console where it is filtered and amplified for display to the operator. The same console and wires can transmit electrical energy through the cable, the handle of the catheter, the insulated wires in the catheter and the electrode to the tissue. This electrical energy may be in the form of pulsed field energy which may be transmitted when the catheter is adherent to the tissue to provide additional ablation. Radiofrequency or other energy may also be transmitted through the same route to warm the tissue when cryotherapy has terminated to accentuate tissue ablation or to accelerate the melting of the ice that attaches it to the tissue.

Each cryotherapy element in an example catheter has a dedicated tube for the delivery of coolant and another for the removal of coolant. Each pair of tubes may be connected to a cryotherapy system including a reservoir of coolant and the system of valves, filters and flow regulators that ensure the appropriate delivery of coolant to that cryotherapy element on command in a safe manner. The individual cryotherapy elements have separate control components but may be supplied from the same coolant reservoir and controlled from the same console. Each of the elements can be controlled separately, for example, the distal element may be activated first, then more proximal elements in sequence.

An example of a metallic tip electrode or array of electrodes has a roughened, textured, corrugated or ridged surface to increase surface area so that when ice forms around it in response to cryotherapy delivery, the catheter adheres firmly to the ice and any tissue contained in it, providing an anchor for the catheter. Force applied in a forward direction to the proximal end of the catheter when the tip is adherent to the muscle can push its shaft against a concave surface, whereas traction applied to the proximal end can pull the shaft against a convex surface.

Applying moderate force to the catheter in the manner described (traction, forward force, torque, deflection of catheter, deflection of sheath) while the distal cryotherapy element is firmly anchored to the tissue forces the part of the catheter containing the other cryotherapy elements into firm and continuous contact with the heart wall. Cryotherapy delivered through these elements will produce a long but narrow zone of ablation. As the cryotherapy elements are continuous with each other, the lesions produced by them become confluent with each other including the initial lesion produced by the distal element to form a single line.

A medical device with a catheter and supporting delivery systems may create areas of tissue damage and therefore block of conduction that are long and narrow. The catheter may have an element at the tip consisting of an electrode or array of electrodes through which cryotherapy is delivered to ablate the tissue in contact with its surface and to create adhesion between the catheter tip and the tissue. This distal element may be composed of a variety of metals or other conductors of electrical energy to permit the recording of electrograms from the tissue and the delivery of electrical energy to the tissue. The distal element may have a textured or corrugated surface to enhance its adherence to surrounding ice and tissue. The catheter may have one or more proximal elements through which cryotherapy can be delivered. Cryotherapy delivery through the different elements may be controlled independently. The proximal cryotherapy elements may have circular electrodes forming rings around the catheter which can be used to record electrical activity from the tissue and can be used to deliver electrical energy in the form of radiofrequency energy, pulsed field energy or a combination of energy types. There may be no gap, or just a minimal gap between the 2 or more ablating elements to ensure contiguity of the resulting lesion. Using the adhesion at the tip of the catheter, traction or forward force can be exerted on the catheter to force the proximal elements firmly against the wall of the heart during the delivery of cryotherapy and pulsed field energy. When the catheter lies on a concave surface, traction will be applied; on a concave surface, forward force will be used. The catheter has a uniform soft consistency along its length. The stiffness of the catheter may be modified by the insertion of a variety of stylets and its shape may be alterable by traction on a deflection wire.

What is claimed is:

1. A catheter comprising:
a tip portion that is configured to deliver cryotherapy;
a therapy portion that extends from the tip portion, the therapy portion including one or more segments, each segment configured to deliver cryotherapy; and
a plurality of electrodes disposed on an outer surface of the therapy portion.

2. The catheter of claim 1, wherein the catheter is substantially cylindrical and has a substantially uniform diameter.

3. The catheter of claim 1, wherein the tip portion includes one or more tip electrodes.

4. The catheter of claim 1, wherein the tip portion has a roughened or corrugated surface.

5. The catheter of claim 1, further comprising coolant delivery conduits and coolant removal ducts connected to the tip and to each of the one or more segments.

6. The catheter of claim 1, wherein each of the plurality of electrodes forms a conductive ring disposed circumferentially about the outer surface of the therapy portion.

7. The catheter of claim 1, further comprising a plurality of electrical connections connected to the plurality of electrodes to transmit electrical current to and from the plurality of electrodes.

8. The catheter of claim 1, further comprising a deflection wire that extends through the catheter and is configured to provide adjustable deflection of the tip.

9. A method comprising:
inserting a catheter in a human body, the catheter includes a catheter tip and a therapy portion near the catheter tip;
locating the catheter tip in a heart chamber;
bringing the catheter tip into contact with a heart wall;
reducing temperature of the catheter tip to adhere the catheter tip to the heart wall;
applying a force along the catheter to bring the therapy portion in contact with the heart wall; and
applying therapy to the heart wall from the therapy portion.

10. The method of claim 9, wherein applying therapy to the heart wall includes applying cryotherapy.

11. The method of claim 9, wherein applying therapy to the heart wall includes applying pulsed field energy.

12. The method of claim 9, wherein applying therapy to the heart wall includes applying Radio Frequency (RF) energy.

13. The method of claim 9, wherein applying therapy includes applying cryotherapy along a substantially linear therapy portion to form a substantially linear ablation lesion.

14. The method of claim 9, wherein applying therapy includes applying cryotherapy and subsequently applying Radio Frequency (RF).

15. The method of claim 9, further comprising:
subsequently detaching the catheter tip from the from the heart wall; and
withdrawing the catheter tip from the heart.

16. A catheter comprising:
a tip portion that has a roughened or corrugated surface and is configured to deliver cryotherapy;
a therapy portion that extends from the tip portion, the therapy portion including three or more segments, each segment configured to deliver cryotherapy;
a plurality of coolant delivery conduits and coolant removal ducts connected to the tip and to each of the one or more segments;
a plurality of electrodes, each electrode formed of a conductive ring disposed along an outer surface of the therapy portion; and
a plurality of electrical connections connected to the plurality of electrodes to transmit electrical current to and from the plurality of electrodes.

17. The catheter of claim 16, further comprising a deflection wire that extends through the catheter and is configured to provide adjustable deflection of the tip.
